(19) United States
(12) Patent Application Publication
(10) Pub. No.: US 2002/0150914 A1
(43) Pub. Date: Oct. 17, 2002

(54) Recombinant antibodies from a phage display library, directed against a peptide-MHC complex

(75) Inventors: Peter Sejer Andersen, Copenhagen (DK); Soren Buus, Bronshoj (DK); Jan Engberg, Copenhagen (DK); Lars Fugger, Copenhagen (DK); Anette Stryhn, Bronshoj (DK)

Correspondence Address: BIRCH STEWART KOLASCH & BIRCH, PO BOX 747, FALLS CHURCH, VA, 22040-0747, US

(73) Assignee: Kobenhavns Universitet

(21) Appl. No.: 09/957,113

(22) Filed: Sep. 19, 2001

Related U.S. Application Data
(63) Continuation of application No. 08/981,021, filed on Mar. 20, 1998, now abandoned.
Application No. 08/981,021 is a 371 of international application No. PCT/DK96/00296, filed on Jul. 1, 1996.

(30) Foreign Application Priority Data
Jun. 30, 1995 (DK) 0778/95
Oct. 30, 1995 (DK) 1214/95

(51) Int. Cl. C12Q 1/68; C07H 21/04; C12P 19/34; C12P 21/02; C12N 5/06

(52) U.S. Cl. 435/6; 435/69.1; 435/91.2; 435/326; 435/320.1; 536/23.53

(57) ABSTRACT

The invention relates to a method of producing an antibody or an antibody fragment specifically recognizing a peptide-MHC complex. It also relates to antibodies and antibody fragments according to the invention that are conjugated to a pharmaceutical or to a superantigen. The invention relates to a pharmaceutical composition comprising antibodies or antibody fragments a according to the invention for the prevention or treatment of infectious and autoimmune diseases, cancer and to compositions for the diagnosis of said diseases.

### FIELD OF INVENTION

Specific recognition of peptide-MHC complexes by the T cell receptor is a key reaction in the specific immune response. Antibodies against peptide-MHC complexes would therefore be valuable tools in studying MHC function and T cell recognition, and could lead to novel approaches in diagnostics and immunotherapy. However, it has proven difficult to generate antibodies or fragments of antibodies with the specificity of T cells by conventional hybridoma technology. The present invention relates to recombinant antibodies and fragments of antibodies with the antigen-specific, MHC-restricted specificity of T cells.

### BACKGROUND OF THE INVENTION

T and B cells represent two fundamentally different recognition modes of the specific immune system. Through alternating selection processes T cells are educated to recognize antigenic peptides presented in association with self-molecules of the major histocompatibility complex (MHC) on the surface of antigen presenting cells. In contrast, B cells are not educated to be self-MHC restricted and B cell receptors (antibodies), whether soluble or in membrane bound form, recognize three-dimensional target structures.

The distinctly different education of B and T cells explains why antibodies with the MHC-restricted specificity of T cells are rarely occurring and why it has been difficult to generate such antibodies using conventional B-cell hybridoma technology. Only a few and sporadic publications have reported the generation of self-MHC restricted antibodies by conventional means (Wylie et al., 1982; van Leeuwen et al., 1979; Froscher et al., 1986), whereas others have reported that their attempts have failed (Tamminen et al., 1987; Rubin et al., 1989). More recently, a few anti-peptide-MHC antibodies have been reported (Murphy et al., 1989; Duc et al., 1993; Aharoni et al., 1991). The molecular basis for the past difficulties may be found in the recently solved structures of peptide-MHC class I complexes (Fremont et al., 1992; Madden et al., 1993; Young et al., 1994). Peptides are deeply buried inside the MHC and are presented as extended mosaics of peptide residues intermingled with self-MHC residues (op.cit.). Strikingly, no more than 100-300 A² of MHC class I bound peptides are facing outwards and thus available for direct recognition (Fremont et al., 1992; Young et al., 1994). Self-MHC is thought to account for the majority of the peptide-MHC complex surface presented to T cells (Fremont et al., 1992; Davis et al., 1988). Antibodies recognizing protein molecules engage about 800 A² of their ligand (reviewed in Davies et al., 1990), and antibody recognition of a peptide-MHC complex would therefore also have to be dominated by self-MHC. Moreover, antibodies are not selected for being self-MHC restricted, rather such antibody specificities might be deleted or silenced (Nemazee et al., 1989) explaining why it is so difficult to raise peptide-MHC specific antibodies. T cells are faced with the same problem of recognizing a ligand dominated by self-MHC and an entire organ has been devoted to educating T cells. Positive and negative selection processes delete the vast majority of maturing thymocytes and only a small minority enters the circulation as mature non-auto-reactive, yet self-MHC-restricted, T cells (von Boehmer et al., 1989).

Faced with the problem of isolating rare antibody specificities, the selection power of the phage display technology (McCafferty et al., 1990; Clackson et al., 1991; Barbas III et al., 1991; reviewed in Winter and Milstein 1991, Winter et al., 1994) is particularly helpful. This technology uses either libraries that have been enriched for the presence of the desired specificity through immunization procedures or libraries of such vast diversity and size that they are likely to contain antibody specificities for any conceivable epitope (Griffiths et al., 1994). To determine whether a selected immunization protocol has worked, it is important to note that the early immune response is dominated by antibodies generated from germline variable genes with affinities in the micromolar range, while increasing somatic hypermutations result in affinities in the nanomolar range (Foote and Milstein, 1991). Similarly phage display antibody fragments isolated from immunization based libraries in general have thousand fold higher affinities than those isolated from naive non-immunized libraries (Marks et al., 1991; Bye et al., 1992; Hoogenboom and Winter, 1992). WO 95/15982 relates to a method for generating an antibody specific for an immunorecessive epitope using the phage display technology.

WO 91/12332 (Kourilsky et al.) discloses restricted monoclonal antibodies characterized by their ability to specifically recognize a complex consisting of a peptide which is characteristic of a pathogenic agent antigen or a cell derangement, and a Major Histocompatibility Complex (MHC) molecule, the antibodies being restricted by not having the ability to recognize said peptide combined with a non peptide specific haplotype MHC molecule.

DE 42 24 542 A1 (Hammerling) outlines a potential method of producing monoclonal antibodies directed against a major histocompatibility complex class I molecule loaded with a peptide antigen which comprises (a) isolating a major histocompatibility class I molecule; (b) inserting a gene coding for the major histocompatibility molecule into the genome of a mouse to express the gene; (c) loading the major histocompatibility class I molecule with the peptide; (d) immunizing the transformed mouse with the loaded molecule; (e) isolating spleen cells from the immunized mouse; and (f) producing monoclonal antibodies against the major histocompatibility complex class I molecule loaded with the peptide by conventional means.

Although WO 91/12332 and DE 42 24 542 A1 both contemplate that it may be possible to produce restricted antibodies by recombinant means, their teachings are speculative, vague and do not enable the person skilled in the art to produce such antibodies.

### SUMMARY OF THE INVENTION

The present invention provides a method to generate antibody and antibody fragments recognizing specific, predetermined peptide-MHC complexes by use of the phage display technology. The speed and feasibility of this technique make it realistic to produce antibodies and fragments of antibodies to a variety of specific peptide-MHC complexes which are contemplated to be useful in studying MHC-restricted T cell recognition and lead to novel approaches in diagnostics and immunotherapy. Furthermore, the invention encompasses recombinant antigen-specific MHC-restricted antibodies as such.

### DETAILED DESCRIPTION OF THE INVENTION

One important aspect of the invention is a method to generate recombinant antibody or antibody fragments recognizing a predetermined peptide-MHC complex by taking advantage of the high selection power of the phage display technology.

In its broadest aspect, the invention relates to a method of producing an antibody or an antibody fragment specifically recognizing a peptide-MHC complex, the method comprising

(a) providing a phage library wherein at least one nucleic acid fragment encoding an antibody or an antibody fragment is expressed

(b) selecting at least one phage producing an antibody or an antibody fragment specifically recognizing the peptide-MHC complex and being encoded by said expressed at least one nucleic acid fragment, and

(c) introducing the at least one nucleic acid fragment encoding said antibody or antibody fragment of said phage into a cell and allowing said cell to produce, as an expression product, said antibody or antibody fragment specifically recognizing said peptide-MHC complex.

By the term “nucleic acid” is meant a polynucleotide of high molecular weight which can occur as either DNA or RNA and may be either single-stranded or double-stranded. The nucleic acid fragment(s) can be obtained either from “teraphage” libraries or from antibody libraries from immunized animals as detailed below.

By a teraphage library is meant a library comprising a very large number of human antibody gene fragments thus providing a high likelihood that a gene fragment encoding an antibody or antibody fragment of interest is present; examples of such very large libraries are described e.g. in Waterhouse, P. G. et al., 1993, Griffiths, A. et al., 1994. This approach will be very useful if treatment of individual patients with one or more antibodies which are specifically reacting with MHC-peptide complexes present within said patient is to be accomplished since it will be possible within a reasonable time to generate specific antibodies directed against a preselected MHC-peptide complex which may be unique for said individual.

A presently preferred embodiment of the method of producing an antibody or an antibody fragment specifically recognizing a peptide-MHC complex comprises the following steps:

(a) isolating RNA from an animal which has been immunized with a peptide-MHC complex,

(b) amplifying at least one nucleic acid fragment encoding an antibody or antibody fragment using the RNA isolated in step

(a) as starting material in a reaction wherein nucleic acids encoding an antibody or antibody fragment are amplified and producing a phage library expressing said nucleic acid fragments

(c) selecting at least one phage producing an antibody or an antibody fragment specifically recognizing the peptide-MHC complex and being encoded by said amplified at least one nucleic acid fragment, and

(d) introducing the at least one nucleic acid fragment encoding said antibody or antibody fragment of said phage into a cell and allowing said cell to produce, as an expression product, an antibody or an antibody fragment specifically recognizing said peptide-MHC complex.

This approach is described in detail in the examples with respect to Fab fragments. The fact that pSAN13.4.1 described in detail in the present application was isolated from a relatively small sized library of about 10⁷ genes and yet is highly specific with an affinity in the nanomolar range, strongly indicates that the mice used for immunization indeed responded to the Ha₂₅₅₋₂₆₂-K^{k} complex by generating high affinity antibodies. Thus, the selected immunization protocol most likely was an important factor in the generation of Fab 14.3.1.

It is evident for the person skilled in the art that a similar approach as outlined in the examples or modified by use of the teraphage technique described in Waterhouse, G. P. et al. can be used with respect to other antibody fragments or entire antibodies. Although the use of the phage display technique is preferred, it is contemplated that recombinant peptide-MHC specific antibodies may also be prepared by use of alternative methods, e.g. single chain Fv antibody fragment display on plasmids (Cull et al., 1992) or cells (Francisco et al., 1993).

In the present specification and claims, antibodies are glycoproteins which exhibit binding specificity to a specific antigen. Antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has a variable domain (V_{H}) at one end followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains.

The term “variable” refers to the fact that certain portions of the variable domains differ extensively in sequence among antibodies and are used in the binding and specificity of each particular antibody for its particular antigen. The variability is concentrated in three segments called complementarity determining regions (CDRs) or hypervariable regions both in the light chain and the heavy chain variable domains. The more highly conserved portions of variable domains are called the framework (FR). The CDRs in each chain are held together in close proximity by the FR regions and, with the CDRs from the other chain, contribute to the formation of the antigen binding site of antibodies (see Kabat et al., 1987). The constant domains are not involved directly in binding an antibody to an antigen, but exhibit various effector functions, such as participation of the antibody in antibody-dependent cellular toxicity.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen. “Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the V_{H}-V_{L} dimer. Collectively, the six CDRs confer antigen binding specificity to the antibody. However, even a single variable domain (or half of an Fv comprising only three CDRs specific for an antigen) i.e. an scFv, has the ability to recognize and bind antigen, although at a lower affinity than the entire binding site. The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. F(ab′)₂ antibody fragments originally were produced as pairs of Fab′ fragments which have hinge cysteines between them.

The “light chains” of antibodies from any vertebrate species can be assigned to one of two clearly distinct types, called kappa (κ) and lambda (λ), based on the amino acid sequences of their constant domains. Depending on the amino acid sequence of the constant domain of their heavy chains, antibodies can be assigned to different classes of which there are five major classes: IgA, IgD, IgE, IgG and IgM. Several of these may be further divided into subclasses (isotypes), e.g., IgG-1, IgG-2, IgG-3, and IgG-4; IgA-1 and IgA-2.

The term “antibody and antibody fragments” is used in the present specification and claims in the broadest sense and specifically covers single monoclonal antibodies as well as antibody fragments (e.g., Fab, Fab′, F(ab′)₂, Fv and scFv), as long as they specifically recognize a peptide-MHC complex. The invention thus relates to methods according to the invention wherein the antibody fragment is selected from the group consisting of Fab, Fab′, F(ab′)₂, Fv, scfv and other antigen-binding subsequences of an antbody. Complexes of the antibodies or fragments of antibodies of the invention, such as IgM or IgG complexes are also within the scope of the invention.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Humanized” forms of non-human (e.g., murine) antibodies are chimeric antibodies or fragments thereof (such as Fv, Fab, Fab′, F(ab′)₂ or other antigen-binding subsequences of antibodies, e.g. scfv) which contain minimal sequence derived from non-human antibody. For the most part, humanized antibodies are human antibodies (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human antibody are replaced by corresponding non-human residues. Furthermore, a humanized antibody may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human antibody and all or substantially all of the FR regions are those of a human antibody consensus sequence. Optimally, the humanized antibody optimally will also comprise at least one portion of an antibody constant region (Fc). For further details, see Winter and Milstein, 1991, or WO 94/29451 which describes humanized antibodies and methods for their production, said antibodies having specificity for the epitope recognized by the murine monoclonal antibody L243.

When an antibody or a fragment of an antibody specifically recognizing a peptide-MHC complex has been produced by the method of the invention, the person skilled in the art will be able to prepare appropriate analogues of said antibody or antibody fragment, e.g. “humanized” antibodies comprising an antigen-binding subsequence prepared by the method of the invention and FR regions of e.g. a human antibody consensus sequence as well as at least a portion of an antibody constant region, if appropriate. As will be evident from the above outline of the art of production of “humanized” antibodies, the possibilities of variation are numerous and the person skilled in the art will be able to make appropriate choices depending on the purpose of the particular antibody.

The human major histocompability complex (MHC), also designated the human leucocyte antigen (HLA) system was 35 initially characterized using maternal antisera that identified paternal transplantation antigens expressed in the offspring. HLA typing was originally developed to facilitate organ and tissue transplantation, particularly renal transplantation. Two classes of alleles are found, HLA class I alleles and HLA class II alleles. The most commonly expressed HLA genes are among the most polymorphic loci in the genome, with the polymorphism concentrated in the peptide binding site.

Among the 15 detected class I genes, only three—the HLA-A, HLA-B and HLA-C loci—constitute the core of the socalled classic HLA class I genes. These genes are highly polymorphic, are expressed on virtually all nucleated cells, and are known, at least in the case of HLA-A and -B, to restrict T-cell responses to intracellular antigens. Presently, a total of 125 alleles of the HLA-A, -B and -C genes have been officially acknowledged by the WHO Nomenclature Committee for Factors of the HLA System.

In order to be assembled with β₂ microglobulin and transported to and expressed on the cell surface, an HLA class I molecule requires the presence of peptides that bind that specific HLA allele. The peptides are provided by a process termed antigen processing. All the peptides investigated contain at least two “anchor positions” in which only a single amino acid or a few amino acids with closely related side chains have been detected. Invariably, one of these anchors was the C-terminal position. In the natural environment, the peptide generally has a length of 8 to 10 residues. (Madden, D. R., 1995).

HLA class II molecules are structurally highly related to class I molecules; this relatedness includes the domain organization and probably also the antigen-binding site. Class II molecules are predominantly involved in the interaction between specific cell types that regulate immune responses, among these B cells, activated T cells, dendritic cells, and cells of the myelomonocytoid lineage (macrophages). Three isotypes, called DR, DQ, and DP, are distinguished among the class II proteins. Because of the strong linkage disequilibrium between DR, DQ, and often also HLA-B genes, a specific DR allelle in an individual of a given ethnic origin will often indicate a whole set of DR/DQ/B alleles; the term “DR haplotype” is used to describe this typical set of class II (and sometimes class I) allelles. Initial data providing insight into the nature of the peptides that are found to be bound to class II molecules suggest that they tend to be longer and less restricted than the peptides that bind to class I molecules, e.g. 13-17 amino acids. (Madden, D. R., 1995).

In the present specification and claims, the term “peptide” comprises both short peptides with a length of at least two amino acid residues and at most 10 amino acid residues and oligopeptides (11-100 amino acid residues) as well as proteins (the functional entity comprising at least one peptide, oligopeptide, or polypeptide which may be chemically modified by being glycosylated, by being lipidated, or by comprising prosthetic groups). The definition of peptides also includes native forms of peptides/proteins in animals including humans as well as recombinant proteins or peptides in any type of expression vectors transforming any kind of host, and also chemically synthesized peptides.

The term “an MHC molecule” is explained above. The term “a peptide-MHC complex” means a peptide which is capable of binding to a particular MHC molecule thereby establishing a specific peptide-MHC complex. It will be evident from the above that it is possible to produce numerous different antibodies by the method of the invention by making appropriate variations with respect to the particular peptide and MHC molecule used to generate or select the antibody in question.

The term “specifically recognizing a peptide-MHC complex” means that the antibody is capable of binding to the peptideMHC complex with an equilibrium dissociation constant, Kₒ in the range of 10⁻⁷ to 10⁻¹⁰ M. The equilibrium dissociation constant, K_{D}, of an antibody or fragment thereof produced by the method of the invention may be determined by any appropriate method as selected by the person skilled in the art, e.g. by the method outlined in Example 3, wherein the kinetic binding analysis of pSAN 13.4.1 by use of surface plasmon resonance is described in detail.

An important step in the method of the invention is the selection of a phage expressing an antibody or antibody fragment specifically recognizing a peptide-MHC complex. In presently preferred embodiments, this selection is performed by incubating cells expressing the peptide-MHC complex with the phage library and/or incubating the phage library with beads to which the peptide-MHC complex is bound. In more general terms, the selection is performed by panning a library of phages expressing antibody or antibody fragments on alternating matrices carrying the peptide-MHC complex as the common denominator. In a preferred embodiment, the alternating matrices are cells expressing the peptide-MHC complex and beads to which the peptide-MHC complex is bound. Alternatively, two different plastic surfaces such as immunotubes (NUNC) or latex beads may be used as matrices coated with the selected MHC/peptide complex. Further, it may be suitable that bound phages are eluted and amplified in a cell such as E. coli after each panning round and optionally analyzed by FACS for binding to cells expressing the MHC molecule in complex with said peptide.

It is contemplated that one important reason for the success of the present approach is the selection power of the phage display technology for recombinant antibody generation allowing very large antibody libraries to be screened efficiently and within a short time. Furthermore, purified MHC molecules that had been enriched for one particular peptide were used in the immunization as well as the selection procedure. It has been calculated that about 80% of the selected MHC molecules were loaded with the desired peptide, i.e. 0.8 mol specific peptide per 1 mol MHC molecule when the procedure described in the examples of the present application has been used. It is contemplated that this enrichment is also an important factor for the success of the immunization as well as the subsequent pannings and thus, it may be advantageous to further enrich the MHC molecules e.g. up to about 90%, about 95% or even about 98% or about 99%. i.e. substantially 100% specific peptide-MHC loading. If the immunization step is avoided by use of the teraphage technique, it is contemplated that it is even more important for a successful selection procedure that purified MHC molecules which have been enriched for one particular peptide is used. However, it is not unlikely that beneficial results may also be obtained with less enrichment of selected MHC molecules with the desired peptide, e.g. about 20%, 30%, 50%, 66% or 75%.

The method of the invention further comprises a step wherein the nucleic acids encoding an antibody or antibody fragment are introduced into an appropriate cell so as to produce an antibody or an antibody fragment specifically recognizing the desired peptide-MHC complex. The cell can be selected from the group consisting of a microorganism such as a bacterium, e.g. Escherichia coli, a yeast, a protozoan, and a cell derived from a multicellular organism such as a fungus, an insect cell, a plant cell, a mammalian cell or a cell line. Alternatively, the DNA encoding the ScFv fragment of the antibody could be introduced into embryonic stem cells to generate transgenic animals producing the antibody fragment under control of tissue specific promoters (Logan, 1993).

Irrespective of the type of organism used, the DNA fragment comprising the nucleic acids encoding the antibody or fragment of antibody is introduced into the organism or cell either directly or by means of a suitable vector. The cells producing the desired antibodies or fragments of antibodies are then selected based on levels of productivity under conditions suitable for the vector and the cell. The selected cells are grown further and form a very important and continuous source of the desired antibodies or antibody fragments.

If considered appropriate, the DNA fragment encoding an antibody or antibody fragment can be cloned, and the nucleotide sequence determined. If desired, the person skilled in the art can then by using conventional techniques such as e.g. site-directed mutagenesis or random mutagenesis in the CDR-region prepare nucleic acids encoding novel antibodies or fragments thereof with the same specificity as the antibody having the originally determined nucleotide sequence, but with altered thermodynamic characteristics (kₐ, k_{d}, K_{D} etc.). The selection of these novel antibodies requires the use of a selection procedure essentially as outlined with respect to the original antibody. In a similar manner, it will be possible to prepare genetically engineered antibodies with a binding specificity towards another peptide-MHC complex, if the selection procedure is appropriately amended.

In a preferred embodiment, the invention relates to a method wherein the nucleic acids encoding said antibody fragment is encoded by the plasmid pSAN 13.4.1 which has been deposited on Jun. 28, 1995 with the collection of Deutsche Sammlung von Mikroorganismen und Zellkulturen GmbH (DSM) under the accession number DSM 10070 in accordance with the provisions of the Budapest Treaty.

One important embodiment of the invention thus relates to a DNA fragment or an analogue or subsequence thereof encoding a restricted antibody or antibody fragment of the invention contained within an expression vector which is capable of replicating in a host organism or a cell line. The vector may in particular be a plasmid, a phage, a cosmid, a mini-chromosome or a virus. In an interesting embodiment of the invention, the vector may be a vector which, when introduced in a host cell, is integrated into the host cell genome.

In another aspect, the invention relates to an antibody or antibody fragment specifically recognizing a peptide-MHC complex prepared by the method of the invention. In particular, the invention encompasses a non-glycosylated antibody or antibody fragment specifically recognizing a peptide-MHC complex. Such an antibody or antibody fragment may be produced when the plasmid encoding the antibody or antibody fragment is expressed in E. coli.

Within the scope of the invention is an antibody or antibody fragment according to the invention conjugated to a pharmaceutical agent, such as a pharmaceutical agent selected from the group consisting of antibiotic, cytotoxic and antineoplastic agents. Alternatively, the antiody or antibody fragment may be conjugated to a superantigen, which is capable of activating T lymphocytes, such as the bacterial superantigen staphylococcal enterotoxin A (SEA) (Dohlstein M. et al., 1994). Superantigenicity may be conveyed on cells recognized by the antibody and thereby evoke T cells to suppress tumour growth or autoimmune disease as described in further detail in the following. In another approach, the antibody or antibody fragment is conjugated to a polymerized carbohydrate, which is capable of activating complement and thereby targeting desired cells expressing the peptide-MHC complex recognized by the antibody or antibody fragment.

For some purposes, it may be an advantage that the antibody is a bifunctional antibody which contains one combining site specifically recognizing a specific peptide-MHC complex and another site specifically recognizing an epitope of another antigen, e.g. an epitope on a cytotoxic T-cell, or a pharmaceutical agent.

The bifunctional antibody may be produced by hybrids between two monoclonal cell lines producing the two relevant antibodies or may be produced by chemically linking fragments of the two antibodies.

The term “combining site” is understood to mean the antigen recognition structure in the variable region of the antibody molecule. Bifunctional antibodies make possible special procedures for detecting the peptide-MHC complex in a sample and for targeting a pharmaceutical agent, a biologically active molecule or another antigen to the site where the reagent has the greatest effect.

In an advantageous embodiment, the other antigen with which the bifunctional antibody is reactive is an effector cell such as a differentiation antigen of cytotoxic T-cells (cf. Staerz et al., 1985; van Ravenswaay-Claasen et al., 1993; Fanger et al., 1989). The pharmaceutical agents with which the hybrid antibody may be reactive is preferably selected from cytotoxic, antibiotic or antineoplastic agents (cf. Coller and Kaplan, 1984).

Clinically, new ways of manipulating T cell responses may be devised by the use of antibodies and fragments of antibodies of the invention. Intracellularly located viruses, bacteria, or parasites are normally presented by MHC class I and recognized by cytotoxic T cells rather than by antibodies. Antibodies that are specific for peptides presented in complex with MHC class I molecules could be conjugated to toxins and used to mimic T cell responses eradicating e.g. virus infected cells where T cells had failed to do so. An example of a disease where a such approach could be useful is HIV. These antibodies can also direct complement lysis against T cell ligands suggesting other novel approaches to immunotherapy.

The idiotypic (antigen binding) structure of an antibody is antigenic and can thus give rise to specific antibodies directed against the idiotypic structure. The antibodies raised against the idiotype are called anti-idiotypic antibodies. Such antibodies may mimic the structure of the original antigen and therefore may function as the original antigen. Such antibodies may be able to substitute the original antigen for a part or all of the functions, usability and properties. Thus, within the scope of the present invention is an anti-idiotypic antibody which is directed against an antibody fragment according to the invention.

The antibody may also be an anti-anti-idiotypic antibody directed against an anti-idiotypic antibody which is an antibody directed against the site of an antibody which is reactive with the epitope on the antigen. The anti-idiotypic antibody may be prepared by a methods well known to the person skilled in the art. Also anti-anti-idiotypic antibodies which are directed against the anti-idiotypic antibody described above are within the scope of the invention.

Thus, the invention also provides a pharmaceutical composition for combatting intracellularly located pathogens selected from the group consisting of viruses, bacteria and parasites, said composition comprising an antibody or fragment according to the invention and a pharmaceutically acceptable excipient. The pharmaceutical compositions described above may comprise a monoclonal antibody or a mixture of monoclonal antibodies specifically recognizing the peptide-MHC complex, or a mixture of antibodies recognizing different peptide-MHC complexes.

It has been found that susceptibility to a wide variety of diseases is preferentially increased in individuals of particular HLA genotypes. There are now more than 30 diseases associated with HLA genotype, and more than 35 to 50 expressed genes that map in the HLA region. The HLA class I and II molecules function by binding antigenic peptides and presenting them to CD8+ and CD4+ T cells, respectively. It is therefore very likely that certain HLA alleles confer susceptibility to autoimmune diseases at the molecular level by binding specific antigenic peptides that other alleles do not bind. For further information see: Fugger L. et al., 1995.

Alternatively, anti-peptide-MHC antibodies may block inappropriate immune T cells responses such as those leading to auto immunity (Aharoni et al., 1991). MHC being a significant risk factor in many autoimmune diseases indicates the existence of a single or a few diseases initiating self-MHC restricted autoantigen. If so, antibody fragments directed against such ligands could prove powerful inhibitors of in vivo autoimmune T cell responses.

The generation of blocking antibodies would require knowledge about the disease inducing T cell epitope and the restricting MHC molecule, requirements that may be fulfilled in the future (Tisch et al., 1993; Kaufman et al., 1993; Michaelsson et al., 1994; Wurcherpfennig and Strominger, 1995).

In a further embodiment, the invention thus relates to a pharmaceutical composition for blocking an inappropriate T cell response comprising an antibody or antibody fragment according to the invention and a pharmaceutically acceptable excipient. In particular the invention relates to a pharmaceutical composition for prevention after establishment of being in a high risk group of developing a malignant tumour, treatment or prevention of a relapse of a malignant tumour, said composition comprising an antibody or antibody fragment according to the invention and a pharmaceutically acceptable excipient. The pharmaceutical compositions may be formulated by methods well known within the art.

Another aspect of the invention relates to a pharmaceutical composition for treatment or prevention of a relapse of an autoimmune disease, said composition comprising an antibody or antibody fragment according to the invention and a pharmaceutically acceptable excipient.

In a further aspect, the invention relates to the use of a pharmaceutical composition according to the invention for the prevention or treatment of a disease selected from the group consisting of HLA class I associated diseases (ankylosing spondylitis, Reiter disease, psoriatic spondylitis, idiopathic hemochromatosis, psoriasis vulgaris and Behcet disease) and HLA class II associated diseases (rheumatoid arthritis, pauciarticular juvenile rheumatoid arthritis, systemic lupus erythematosus, Sjogren disease, IDDM, Addison disease, Graves disease, Hashimoto disease, celiac disease, primary biliary cirrhosis, pemphigus vulgaris, epidermolysis bullosa acquisita, Hodgkin disease, cervical squamous cell carcinoma, multiple sclerosis, optic neuritis, narcolepsi, myasthenia gravis, Goodpasture syndrome and alopecia areata). Furthermore, the invention relates to the use of a pharmaceutical composition according to the invention for combatting intracellularly located pathogens selected from the group consisting of viruses, bacteria and parasites by directing complement lysis.

Antibodies directed against specific peptide-MHC combinations may be used to detect the presence of such peptide-MHC complexes on the surface of cells in the body. The requirements for the generation of peptide-MHC complexes are that the cell express the MHC in question and that it can generate the peptide in question. The former depends on the cell type and whether the cell has been stimulated with various cytokines. In general, all somatic cells express MHC class I, whereas a more restricted set of immune cells (dendritic cells, B cells etc) express MHC class II. As an example of stimulated MHC expression one could mention the γ-interferon induced MHC class II expression on macrophages. The latter depends on the pool of proteins available to the cell and the cellular machinery for antigen degradation. In general, antigen processing generates short peptide fragments from all intracellular proteins including mutated self-proteins and protein antigens derived from infectious agents. Thus, the MHC molecule binds and presents peptides derived from all self and foreign protein antigens.

The normal function of T cells is to scrutinize the identity of the peptide-MHC complexes presented by cells in the body for the purpose of attacking foreign combinations and ignoring self combinations. An antibody, which can recognize the identity of peptide-MHC complexes would be able to make the same self-non-self distinction. Such an antibody would have several advantages compared to T cells some of which are: antibodies are easier to prepare and label, they are easier to transport and store, they are thought to have higher affinities allowing more sensitive and robust assay, they can be tested in various non-physiological buffers. With peptide-MHC specific antibodies it would be possible to detect the presence of mutated self-proteins (eg mutated oncogenes) or proteins derived from foreign infectious agents (eg. virus) whether they are based intra- or extra-cellularly. In particular, intracellularly based antigens, which normally would escape antibody detection, can be detected using such “T cell-like” antibodies.

Thus, peptide-MHC specific antibodies have a diagnostic potential since they can identify—by way of example, but not limited to—tumor cells harbouring mutated oncogenes or infected cells harbouring virus's. Detection could be performed by fluoresence activated cell sorting (FACS, such as FACS Star from Becton Dickinson) which would allow detection, quantitation, characterization and purification of the cells of interest. The peptide-MHC specific antibodies could also be attached to magnetic particles for high capacity cell sorting and purification of cells of interest. For high sensitivity detection with the potential for high capacity throughput one could label peptide-MHC specific antibodies (eg. with biotin) and perform enzyme linked immunosorbent assays (ELISA's) with the antigen coupled directly to microtiter plates or via another antibody (the latter known as a sandwich technique).

Cells from the blood or from tissue obtained through biopsies (live or fixed) can be tested. Recent years has established that MHC can be shed by cells into the blood stream (see Puppo et al, 1995). Concentration of soluble MHC up to 0.5-25 microgram/ml has been reported. The concentration increases locally around sites of inflammation. Experiments have shown that these peptide-MHC complexes have retained their W6/32 epitope (W6/32 is a monoclonal anti-MHC class I antibody, ATCC HB95). This epitope depends on the proper binding of both beta-2 microglobulin and peptide to the MHC class I. Thus, one can conclude that these soluble MHC molecules represents peptide-MHC complexes and that circulating soluble peptide-MHC complexes carries an imprint of the total intracellular protein metabolism of the body. These specificities can be accessed simply by taking a blood sample and making an ELISA using peptide-MHC specific antibodies for detection. This will have significant diagnostic implications since it will allow the intracellular metabolism be assessed using a blood sample.

The availability of peptide-specific, MHC-restricted recombinant antibodies may thus be useful both scientifically and clinically as they due to their soluble nature, high affinity and stability are well suited for detecting the presence of T cell epitopes under conditions (e.g. immunoprecipitations, immuno histochemistry etc.) which precludes using T cells or recombinant soluble T cell receptor. Thus, questions relating to how and where certain events occur during antigen presentation may be addressed directly, and the expression of T cell epitopes on the antigen presenting cell may be visualized and quantified. Clinically, antibodies with peptide/MHC specificity might be useful in diagnostics since mutations or intracellular infections normally reserved for T cell recognition would be available to the antibodies, too. Diagnostic kits relating to the detection of specific T cell epitopes by the use of antibodies (or antibody fragments) are, therefore, dependent on the availability of the antibodies with the specificity described in this invention.

The present invention provides a diagnostic composition for the detection of the presence of a peptide-MHC complex which comprises an antibody or antibody fragment, an anti-idiotypic antibody or an anti-anti-idiotypic antibody according to the invention. In particular, the invention relates to a diagnostic composition for the detection of an intracellularly located pathogen selected from the group consisting of viruses, bacteria and parasites, to a diagnostic composition for the detection of a malignant tumour. Various human tumour antigens recognized by T-cells are known and may constitute useful targets for specific immunotherapy by use of the antibodies and antibody fragments of the invention.

The invention also relates to a diagnostic composition for the detection of the presence of an autoimmune disease and to a diagnostic composition for the assignment of individuals to high risk groups in relation to developing malignant tumours or autoimmune diseases.

### LEGEND TO FIGURES

FIG. 1. Phage selection by biopanning

A library of phages expressing immunoglobulin Fab fragments was panned on alternating matrices carrying Ha₂₅₅₋₂₆₂-MHC complexes as the only common denominator. The first and third pannings were performed on Ha₂₅₅₋₂₆₂ loaded RMA-S·K^{k} cells, and the second and the fourth pannings were performed on beads (Interfacial Dynamics, Oregon) coated with purified Ha₂₅₅₋₂₆₂-K^{k} complexes. Bound phages were eluted and amplified in E. coli after each panning round and analyzed by FACS for binding to peptide loaded RMA-S cells transfected with K^{k}. The peptides were Ha₂₅₅₋₂₆₂ (open bars) or NP₅₀₋₅₇ (closed bars). The black arrow indicates the background signal level. Mean fluorescence intensity=MFI.

FIG. 2. Purification and binding characteristics of Fab 13.4.1

A) Purified and reduced Fab13.4.1 was analyzed by polyacryamide-SDS gel electrophoresis followed by detection using the silver staining method.

(B) FACS analysis of binding of purified Fab13.4.1 to latex beads coated with Ha₂₅₅₋₂₆₂/K^{k} (open circles), NP₅₀₋₅₇/K^{k} (closed circles), K^{k} (open triangles) or Ha₂₅₅₋₂₆₂ (closed triangles). Purified H-2K^{b} molecules complexed with endogeneous peptide were also tested and showed no binding to Fab 13.4.1 (results not shown). Coated Latex beads were incubated with increasing concentration of Fab13.4.1 and analyzed by FACS using FITC conjugated Rabbit anti mouse IgG (DAKO, Denmark) as detecting antibody.

(C) Competition of binding. Latex beads coated with Ha₂₅₅₋₂₆₂/K^{k} were incubated with 3 nM Fab13.4.1 and increasing concentrations of soluble Ha₂₅₅₋₂₆₂/K^{k} complexes (open circles), NP₅₀₋₅₇/K^{k} complexes (closed circles), K^{k} or Ha₂₅₅₋₂₆₂ (closed triangles) followed by FACS analysis as in FIG. 2B.

FIG. 3. BIAcore measurements

BIAcore sensorgrams of HA₂₅₅₋₂₆₂-K^{k} and NP₅₀₋₅₇-K^{k} complexes binding to immobilized (A) Fab 13.4.1 or (B) H100-27R55 (anti-K^{k}) Complexes were diluted to a concentration of 500 nM and passed over the immobilized antibodies for three minutes.

(C) Kinetic binding curves of HA₂₅₅₋₂₆₂-K^{k} binding to Fab 13.4.1. Complexes were applied in increasing concentrations with a three-minute association phase and a six minute dissociation phase. The actual concentrations are shown on the sensorgram. All binding curves are expressed as resonance units (RU) as a function of time.

FIG. 4. Specific inhibition of peptide-specific, MHC-restricted T cell responses

RMA-S·K^{k} cells were incubated overnight at 26° C. with suboptimal doses of Ha₂₅₅₋₂₆₂ or NP₅₀₋₅₇. The peptide loaded RMA-S·K^{k} cells were co-cultured with the Ha₂₅₅₋₂₆₂ specific T cell hybridomas in the presence of graded doses of Fab13.4.1. As a control the NP₅₀₋₅₇ specific T cell hybridomas were co-cultured in the presence (black bars) or absence (white bars) of the maximum dose (390 nM) of Fab13.4.1 (insert). The suboptimal doses used were 6 μM Ha₂₅₅₋₂₆₂ for presentation to HK8.3-6F8 (filled in circles), 0.3 μM Ha₂₅₅₋₂₆₂ for presentation to HK8.3-5H3 (filled in squares), 0.3 μM NP₅₀₋₅₇ for presentation to HK9.5-24, and 3 μM NP₅₀₋₅₇ for presentation to HK9.5-162.

FIG. 5. Panning results of epitope specific selection

Complexes between purified K^{k} and Ha₂₅₅₋₂₆₂ (FIG. 5A) or NP₅₀₋₅₇ (FIG. 5B) were generated and coated to plastic surfaces as described in Example 1a. The figure illustrates the relative enrichment of Fab-phages with specificity to K^{k}/HA₂₅₅₋₂₆₂ complexes (as opposed to K^{k}-NP₅₀₋₅₇ complexes) within the Fab-phage library as a result of succesive rounds of pannings.

FIG. 5A shows that (in the case of the K^{k}/Ha₂₅₅₋₂₆₂ library) 18 out of 20 individually selected clones after the 3rd round of panning bound to the antigen (K^{k}/Ha₂₅₅₋₂₆₂) and that binding was peptide specific in 16 out of these 20 clones. (That is, two out of the 20 clones bound to the K^{k}/Ha₂₅₅₋₂₆₂ antigen in a peptide non-specific manner).

FIG. 5B shows that (in the case of the K^{k}/NP₅₀₋₅₇ library) 7 out of 20 individually selected clones after the 3rd round of panning bound to the antigen (K^{k}/NP₅₀₋₅₇) and that binding was peptide specific in 1 out of these 20 clones.

### EXAMPLES

### Methods

### MHC Purification

The AKR derived lymphoma, RDM-4, was used for K^{k} production as previously described (Olsen et al., 1994). About 1×10¹⁰ cells were resuspended in 100 ml of lysis buffer (PBS, 1% Nonidet P40, 25 mM iodoacetamide, 1 mM PMSF (SIGMA, Cat. no. P-7626), 5 mM sodium orthovanadate) and incubated for 10 minutes at ambient temperature. The lysate was cleared by centrifugation, stored at −80° C. and thawed by incubating overnight at +4° C. before further use. The lysate was filtered several times through Millipore filters (from 8 to 0.45μ) and the K^{k} molecules were purified by affinity chromatography using the monoclonal anti K^{k} antibody 11.4.1 (TIB95, obtained from the American Type Culture Collection, ATCC). The affinity column was prepared by covalently coupling of 11.4.1 to an activated cyanogen bromide Sephadex matrix as described by the manufacturer (Pharmacia). The lysate was passed over the column several times followed by extensive washing: (i) 20 column volumes of PBS, 0.1% SDS, 0.5% Nonidet P-40, 0.02% sodium azide. (ii) 20 column volumes of PBS, 0.05% Nonidet P-40, 0.1% sodium azide. (iii) 20 column volumes of PBS, 0.1% sodium azide. Bound K^{k} molecules were eluted with five column volumes (50 ml) of eluting buffer (0.05 M diethanolamine, 0.15 M NaCl, 0.1% sodium azide, 0.1% sodium deoxycholate, pH 11) to elute bound material which was immediately neutralized with 2.5 ml of 2M Tris-HCl, 0.1% sodium azide, pH 6.3. The eluate were concentrated by vacuum dialysis using collodion bags (Satorius Cat. no. 13200 E). 10 ml of PBS, 1% octylglycoside (Sigma) and 0.1% sodium azide was added when the eluate reached a volume of about 0.5 ml, and the vacuum dialysis continued until a final volume of 1 ml was obtained. The protein concentration of K^{k} was estimated by SDS-PAGE and using the BCA assay.

Human β2-microglobulin was obtained from the urine of uraemic patients and purified to homogeneity by gel filtration and chromatofocusing (Stryhn et al., 1994).

### Peptide Synthesis

The influenza virus derived nucleoprotein peptide (NP₅₀₋₅₇, single letter code: SDYEGRLI) and hemagglutinin peptide (Ha255-262:=FESTGNLI) were synthesized manually on a RaMPS synthesizer (Dupont) using standard FMOC-protection strategy.

### Generation of Peptide-MHC Class I Complexes

15 μM detergent solubilised, affinity purified MHC class I molecules were incubated at 18° C. for 30-48 hours with 44 μM NP₅₀₋₅₇ or Ha₂₅₅₋₂₆₂ peptides in the presence of 5 μM human β2-microglobulin. The reaction mixture contained 1 mM PMSF (Sigma, P-7626), 8 mM ethylenediaminetetraacetic acid (EDTA) (BDH, 10093), 1.2 mM 1.10 phenanthroline (Sigma, P-9375), 69 μM pepstatin A (Sigma, P-4265), 128 μM Na-p-tosyl-L-lysine chloromethyl ketone (TLCK) (Sigma, T-7254), 135 μM Na-p-tosyl-L-phenylalanine chloromethyl ketone (TPCK) (Sigma, T-4376), and 1 mM N-ethylmaleimide (NEM) (Sigma, E-3876) in PBS (0.14 M NaCl/0.01 M sodium phosphate buffer). The final detergent concentration in the reaction mixture was 0.05% NP-40 and the PBS was adjusted with a citrate buffer to pH 5.5 assuring almost complete peptide exchange. Non-bound peptide was removed by Sephadex G25 spun column chromatography as previously described (Olsen et al., 1994).

### Immobilization of Purified MHC-peptide Complexes on Latex Beads

Peptide-MHC complexes were generated as described above. Spin column chromatography was performed using a low detergent concentration (0.05% Nonidet P-40 in PBS). Complexes in low detergent buffer were mixed with 1.5×10⁷ sulphurated latex beads (5μ in size, Interfacial Dynamics Corporation, Batch no. 436) and incubated overnight at +4° C. with gentle shaking. The remaining sites on the beads were blocked with 1% bovine serum albumin in PBS. The beads were washed once and resuspended in 1 ml of FACS buffer (1% BSA, PBS, 0.1% sodium azide) and used for either panning or FACS analysis. The bead preparations were stable for about one week at +4° C.

### Generation of MHC-peptide Complexes on Cells

The RMA-S.K^{k} cell line (donated by W. Ortiz-Navarette) is impaired in the processing of MHC class I molecules due to the lack of peptide transporters which direct peptides from the cytoplasm to the endoplasmatic reticulum where endogenous peptide-MHC complexes are formed. By growing the cells in medium containing high concentration of peptide, almost full surface expression of K^{k} can be achieved. Since this effect is a consequence of the peptide, most K^{k} molecules will be complexed with the added peptide on the cell surface. Thus, peptide loaded RMA-S.K^{k} cells only express the desired peptideMHC complex on their surface. The complexes on the cell surface were formed by incubating 10⁷ cells in 5 ml of growth media containing 0.1 mM peptide overnight at 26° C. Cells were harvested by centrifugation, washed once and resuspended in 1 ml of growth media.

### Immunizations

Inbreed BALB/k mice were immunized i.p. with 1 Human Unit BCG (M. tuberculosis bovis, The State Serum Institute). In a total volume of 0.5 ml, 0.5 mg of K^{k} in complex with HA₂₅₅₋₂₆₂ was mixed with 1 mg of PPD (supplied by The State Serum Institute) in a 0.1 mM phosphate buffer (pH 7.5) containing 0.05% Nonidet P-40. An equal volume of a glutaraldehyde solution (0.2% glutaraldehyde, 0.1 mM NaHPO₄, 0.05% Nonidet P-40, pH 7.5) was added, and the mixture was left for two days at 4° C. with end-over-end mixing. To this mixture was added 12.3 ml of PBS, 6.7 ml of 6 mg/ml Al(OH)₃, and the mixture was mixed thoroughly and used for immunization. Three weeks after the BCG priming, mice were immunized twice with 0.5 ml of the antigenic mixture. The immunizations were performed s.c. with two-week intervals. Spleens were collected ten days after the second immunization.

### Creation of a Phage Library Expressing Immunoglobulin Fusion Proteins

Total RNA was isolated from spleens of two immunized mice according to the method of Chirgwin et al., 1979. First-strand cDNA was primed with oligo-dTₙ₌₁₈ using SuperScript Plus (Gibco BRL) reverse transcriptase in a reaction mixture incubated at 42° C. for one hour. Following heat inactivation of the transcriptase, the cDNA template was ethanol precipitated and used for primary PCR amplifications of the VH, Fd and L-chain genes. The primers used below are described in detail by Ørum et al., 1993. PCR reactions were performed in 100 μl volumes containing dNTP's (0.2 mM), reaction buffer supplied by the manufacturer (Cetus), primers and cDNA. VH reactions contained MVH 1-25 and 5 pmoles of each of the MJH 1-4 primers. Fd reactions contained MVH 1-25 and 6.7 pmoles of each of the MCH γ1, γ2A and γ2B primers. L-chain reactions contained MVK 1-25 and 20 pmoles of the MCK primer. Reaction mixtures were overlaid with mineral oil and kept at 94° C. for 2 minutes. Then 1.5U of Supertaq (HT Biotechnology) or Amplitaq (Cetus) was added and the mixtures cycled 30 times (94° C. 1 minute, 55° C. 1 minute, 72° C. 2 minutes) followed by incubation at 72° C. for 10 minutes. The primary PCR amplification products were purified by agarose gel electrophoresis in combination with GeneClean (Bio101, Inc.) procedures.

Assembly of VH genes with CH1-LINK-D: 100 μl reactions contained buffer and dNTP's as above and 5 ng of purified Fd gene fragments, 20 ng of purified CH1-LINK-D, FabTAG.BACK 1-2 (25 pmoles each) and 50 pmoles FabLINK.FORW. Assembly of Fd genes with LINK-D: 100 μl reactions contained buffer and dNTPs as above and 5 ng of purified Fd gene fragments, 2 ng of purified LINK-D, FabTAG.BACK 1-2 (25 pmoles each) and 50 pmoles of FabLINK.FORW. Assembly of L-chain genes with LINK-D: 100 μl reactions contained buffer and dNTPs as above, 5 ng of purified L-chain genes, 2 ng of purified LINK-D and 50 pmoles of FabLINK.BACK and FabTAG.FOR, respectively. The thermocycling programme was initiated as above, cycled 25 times (94° C. 2 minutes, 55° C. 1 minute, 72° C. 2 minutes) and the assembled product was gel-purified.

Final assembly: 100 μl reactions contained buffer and dNTPs as above and 5 ng of purified VH-CH1-LINK-D fragment or 5 ng of purified Fd-LINK-D fragment were mixed with 5 ng of purified L-chain-LINK-D fragment and 20 pmoles of ASSEMBLY-1 primer. The thermocycling programme was initiated as above, run 25 times (94° C. 1½ minutes, 69° C. 1 minute, 72° C. 2 minutes) and the assembled product was gel-purified.

DNA fragments from final assembly reactions were digested with NotI using 10 U per μg of DNA at 37° C. for 2 hours. Following phenol extractions and ethanol precipitation, the DNA was dissolved in SfiI buffer and incubated under oil at 50° C. for 2 hours with 10 U of enzyme per μg of DNA. The DNA was purified using GeneClean procedures and ligated to GeneClean-purified NotI and SfiI-cut pFAB5c. The 20 μl ligation reaction included 0.5 μg of digested vector, 0.5 μg of insert DNA and was incubated overnight at 15° C. with 1.5 U of T₄-DNA ligase (Amersham). The ligation mix was then purified by phenol extraction and ethanol precipitation followed by resuspension in 20 μl of water. Portions of 2 μl were electroporated into E. coli TOP 10F′ cells (British Biotechnology) using a Bio-Rad E. coli pulser set at 25 μF, 2.5 kV and 200 Ohms. Immediately after the pulse, one ml of freshly made SOC medium (Sambrook et al., 1989) was added and the cells were shaken for one hour at 37° C. Serial dilutions were made and spread on LB-ampicillin plates to obtain an estimate of the size of the total library. The VH and L-chain gene library consisted of 1.6×10⁷ clones and the Fd and L-chain gene library consisted of 6×10⁶ clones. Each 1-ml transformation mixture was transferred to 40 ml of 2×TY medium containing 100 μg/ml ampicillin, 8 μg/ml tetracycline and 1% glucose. The cultures were incubated with shaking at 37° C. until an OD₆₀₀ of 1-2 was reached. Aliquots of the libraries were made into glycerol stocks and stored at −80° C. Superinfection of library: 2 liter ml flasks containing 200 ml of 2×TY, 100 μg/ml ampicillin, 8 μg/ml tetracycline were inoculated with 10⁹ transformed cells and shaken at 37° C. at 250 rpm. At an OD₆₀₀ of 0.5, helper phage, R408 (Stratagene) was added at a multiplicity of 50 and infection proceeded for 20 minutes at 37° C. To the cells was then added isopropyl-β-D-thiogalactopyranoside (IPTG) to a final concentration of 0.1 mM, and incubation was continued overnight at room temperature with shaking (250 rpm). Cells were pelleted and the phage supernatant was concentrated by PEG precipitations as described in Ørum et al., 1993 and Engberg et al., 1995.

### Selection of Antigen Binders by Panning

Panning on cells: K^{k} transfected RMA-S cells (RMA-S·K^{k}) loaded with Ha₂₅₅₋₂₆₂ were prepared as described above. 80 μl of cells were mixed with 20 μl of phage library (about 10¹¹ cfu) and incubated at ambient temperature for three hours with gentle shaking. Cells were washed three times with growth media and bound phages were eluted by treatment with 1 mg/ml trypsin (Worthington) for one hour at 37° C. Cells were removed by centrifugation and the supernatant transferred to vials containing 400 μl of exponentially growing E. coli TOP10F′ cells (British Biotechnology) with an OD₆₀₀ of 0.8 to 1.0. The following superinfection was performed as described in the previous paragraph.

Panning on beads: 1×10⁶ Latex beads coated with purified Ha₂₅₅₋₂₆₂-K^{k} complexes were mixed with 20 μl of phage library (about 10¹¹ cfu) in 1% BSA, PBS buffer and incubated for three hours at +4° C. with gentle shaking. Beads were precipitated by centrifugation and washed three times. Bound phages were eluted after the final wash by resuspending the beads in 100 μl of glycine elution buffer (0.1 M glycine-HCl, 0.1% BSA, pH 2.2) and were left for ten minutes at ambient temperature. Beads were removed by centrifugation and the supernatant neutralized by adding 8 μl of 2M Tris-base. The mixture was transferred to vials containing 400 μl of exponentially growing cells, and superinfection was performed as previously described.

### FACS Analysis

FACS analysis was performed to evaluate Fab-phage binding to peptide pulsed RMA-S.K^{k} cells or to detect binding of Fab fragments to various peptide-MHC combinations immobilized on beads.

FACS analysis using cells: 10⁵ RMA-S.K^{k} cells pulsed with peptide were mixed with about 4×10⁹ cfu Fab phages in 100 μl of FACS buffer (1% bovine serum albumin (BSA), PBS, 0.1% sodium azide) or with Fab 13.4.1 and incubated on ice for two hours followed by three rounds of washing with FACS buffer. The detecting antibody was an FITC conjugated goat-antiphage or rabbit antiM13-phage serum (both DAKO, Denmark) used in a 1:100 dilution in FACS buffer. The samples were incubated for 30 minutes on ice. Unbound antibodies were removed by washing and the remaining cells were resuspended in 200 μl of FACS buffer containing 1% formaldehyde and analyzed by FACS.

FACS analysis using beads: Purified Fab 13.4.1 fragments in various concentrations were mixed with 10⁵ beads coated with peptide-MHC complexes. After two hours incubation on ice, the beads were washed tree times followed by 30 minutes of incubation with an FITC conjugated rabbit-anti-mouse IgG serum (DAKO, Denmark) diluted 1:50 in FACS buffer. The samples were further processed as above.

The FACS data were recorded using a FACScan (Becton Dickinson Immunocytometry). Three parameters were collected for each analysis: (i) forward scattered light, (ii) side scattered light and (iii) the fluorescent emissions of fluorescein (515-545 nm). A forward scatter threshold was set to exclude cell or bead debris. Data for 10⁴ beads or cells were collected and analyzed using the Lysis II™ software (Becton Dickinson Immunocytometry).

### Production and Purification of Soluble Fab Fragments from Selected Clone

For the preparation of Fab fragments of pSAN 13.4.1, the Fab coding cassette was transferred to an expression vector which adds an HIS₆-tag to the C-terminus of the light chain in place of the gene III element (Engberg et al., 1995). Using this new construct, Fab fragments were prepared as follows: two 2 l flasks each containing 500 ml of 2×TY, 100 μg/ml ampicillin, 8 μg/ml tetracycline were inoculated with 10 ml of transformed cells and shaken at 37° C. at 250 rpm. At an OD₆₀₀ of 0.5, IPTG was added to a final concentration of 1 mM and incubation was continued for four hours at room temperature with shaking (250 rpm). Periplasmic protein was isolated by resuspending the cells in 10 ml of ice-cold TES buffer (200 mM Tris-HCl, 5 mM EDTA (ethylene diamine tetra-acetic acid di-sodium salt di-hydrate), 500 mM sucrose, pH 8.0). After five minutes 15 ml of 1:4 TES buffer was added and the mixture incubated for 30 minutes on ice. Cellular remains were removed by centrifugation and the periplasmic fraction was dialysed against 4 l of MES buffer (10 mM 2-[N-morpholino]ethanesulphonic acid (SIGMA, Cat. no. M-82-50), 20 mM NaH₂PO₄, pH 5.6) overnight at 4° C. The periplasmic fraction was passed over ABx matrix (J. T. Baker Research Products) and retained material eluted using a gradient of 0.5 M NaH₂PO₄, pH 7.0. Fab fragments were further purified by immobilized metal ion chromatography (IMAC). The Fab-positive fractions from the ABx purification were adjusted to IMAC buffer A (PBS, 0.5 M NaCl) using a five times concentrated buffer stock and applied to a chelating Superose HR 10/2 column (Pharmacia Biosystems) which had been preloaded with 5 column volumes of 0.1 M CuSO₄ followed by column equilibration with buffer A. Bound protein was eluted using a imidazole buffer (IMAC buffer A, 0.5 M imidazole) gradient (0-0.5 M). The amount of purified Fab fragments was estimated by SDS-PAGE and BCA assays. All purifications were performed on a High Load™ system (Pharmacia Biosystems).

### Generation of Class I Restricted Mouse T Cell Hybridomas and Cytotoxic T Cell Assays

The generation and specificity of the class I restricted mouse T cell hybridomas, HK9.5-24 and HK9.5-162 (NP₅₀₋₅₇ specific, K^{k}-restricted (Stryhn et al., 1994), HK8.3-5H3 and HK8.3-6F8 (Ha₂₅₅₋₂₆₂ specific, K^{k}-restricted (Stryhn et al., 1994) have been described.

The K^{k} transfected RMA-S cell line, RMA-S.K^{k} (Stryhn et al., 1994) was incubated overnight at 26° C. with Ha₂₅₅₋₂₆₂, or NP₅₀₋₅₇. After the incubation the cells were washed extensively and resuspended in culture media in varying concentrations of Fab 13.4.1. 10⁵ T hybridoma cells were incubated with 10⁵ loaded RMA-S.K^{k} cells for 24 hours at 37° C. in a total of 250 μl and the supernatant tested for IL-2 release according to Kappler et al., 1981.

### BIAcore Analysis

In order to determine the concentration of complexes formed (i.e. active binding sites of K^{k}), trace amounts of ¹²⁵I-labelled NP₅₀₋₅₇ with known specific activity were added, and complex formation was determined by G-25 spun column chromatography (2). Purified Fab 13.4.1 was diluted to 20 μg/ml in 10 mM acetate buffer, pH 4.5, and coupled to the dextran surface of BIAcore chips by standard amine coupling chemistry according to the manufacturer's recommendations (Pharmacia, Sweden). The amount of Fab fragment coupled to the chips was kept just below 1000 RU. Purified and quantitated peptide/MHC complexes were introduced into the measuring chamber and the flow speed was set to 10 μl/minute to avoid interference of mass transport limitations with the kinetic calculations. All measurements were carried out in PBS containing 0.1% NP-40 at 22° C.

### T Cell Hybridoma Stimulation, T Cell Receptor Specificity

An in vitro stimulation assay was used to determine the fine specificity of the Ha₂₅₅₋₂₆₂/K^{k} specific T cell hybridomas (21). RMA-K^{k} (a K^{k} transfected thymoma), was used as antigen presenting cell. 1×10⁵ RMA-K^{k} per well were incubated with 1×10⁵ of the Ha₂₅₅₋₂₆₂ specific, K^{k}-restricted hybridomas, HK8.3-5H3 or HK8.3-6F8 in a 96 well microtiter plate. Graded concentrations of the Ha₂₅₅₋₂₆₂ peptide, or analog substitutions hereof, were added and the cultures incubated for 24 h in RPMI₁₆₄₀ supplemented with 10% FCS at 37° C. in a humidified atmosphere containing 5% CO₂. Supernatants were harvested and tested for IL-2 content using 4000/well of the IL-2 dependent cell line, HT-2, as originally described by Kappler et al (26). The concentration of peptide needed to obtain 100 Units/ml IL-2 was determined.

### Specificity of the Recombinant pSAN 13.4.1 Antibody

Specific Ha₂₅₅₋₂₆₂-K^{k} complexes were generated, purified, and coated at a suboptimal concentration of 10 pg (10 nM) per well onto Maxisorp (Nunc, Denmark) 96-well flat-bottom microtiter plates. The plates were subsequently blocked in 2% skimmed milk in PBS buffer and then exposed to pSAN 13.4.1 expressed as a fusion protein at the end of pIII of the filamentous phage. The plates were washed, exposed to HRP coupled antiphage antibodies (Pharmacia, Uppsala), washed, developed and read in an ELISA reader at 490 nM. To determine the fine specificity of pSAN 13.4.1, graded concentrations of analog-K^{k} complexes (generated with 450 μM analog to effect K^{k}-saturation) were added as competitors prior to addition of pSAN 13.4.1 phages. The concentration of competitor complex needed to effect 50% inhibition (the IC₅₀) was determined. The lower the IC₅₀ the stronger the binding of the pSAN13.4.1 to the analog-K^{k} complex.

### Example 1

### Generation and Selection of Peptide-specific MHC Restricted Antibodies

Complexes between purified mouse MHC class I, K^{k}, and the K^{k}-restricted Influenza virus derived peptides, Haemaglutinin (Ha₂₅₅₋₂₆₂) were generated as described in the Methods section. For immunization purposes Purified Protein derived Peptide of Tuberculin (PPD) was coupled to the purified complexes and used to immunize Bacille-Calmette-Guerin (BCG) primed H-2^{k} (BALB/k) mice which are largely tolerant to K^{k} as a B cell immunogen, but highly reactive to PPD as a T cell immunogen.

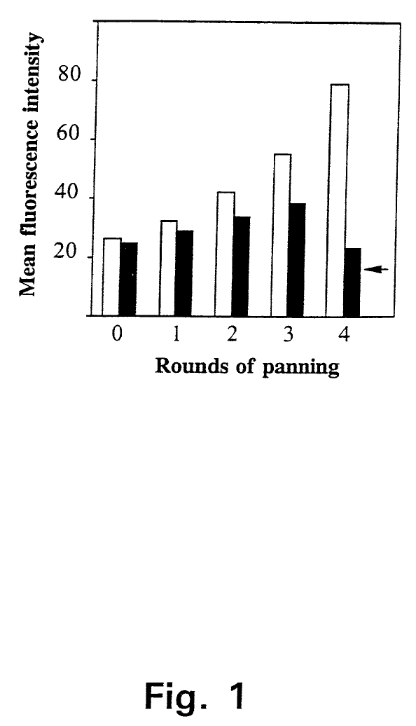

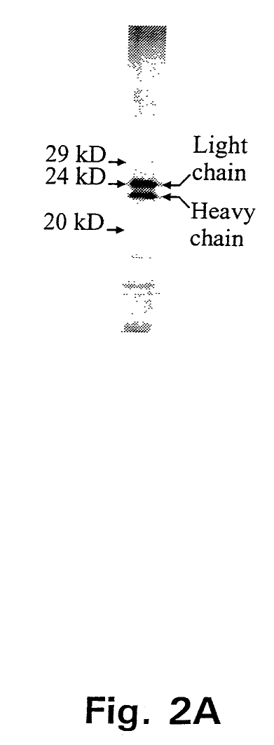

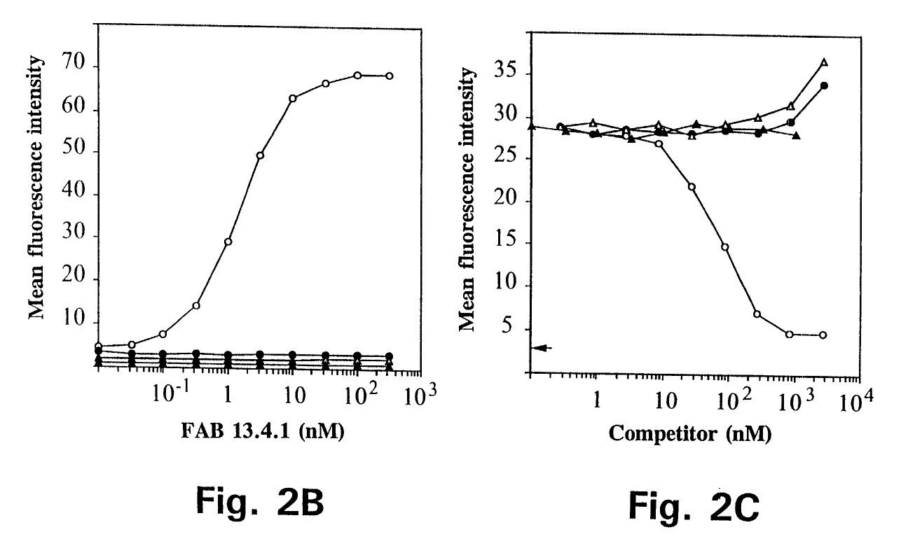

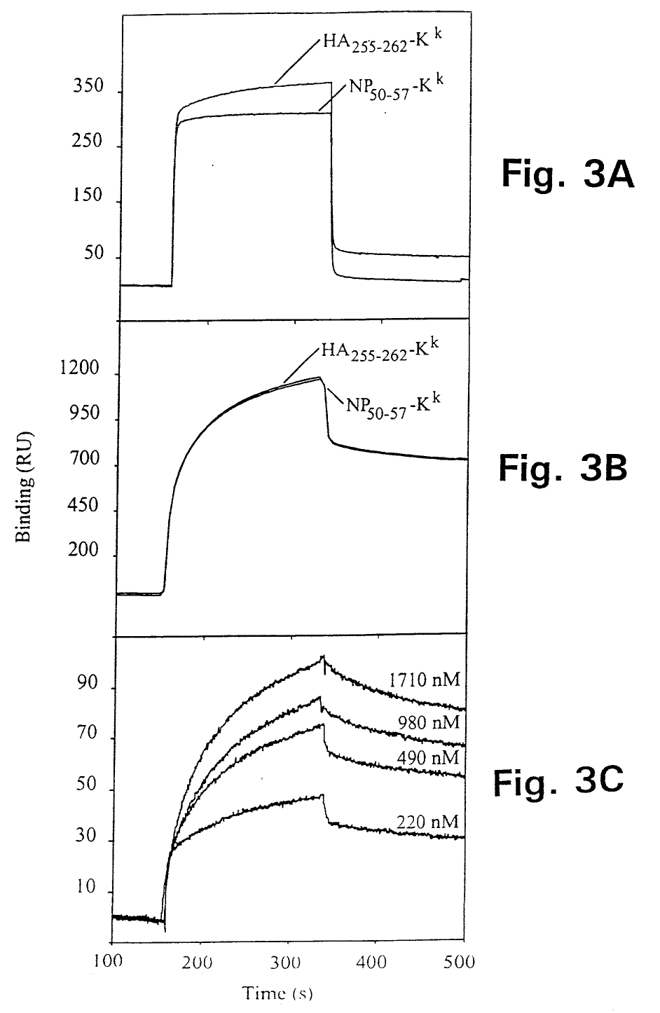

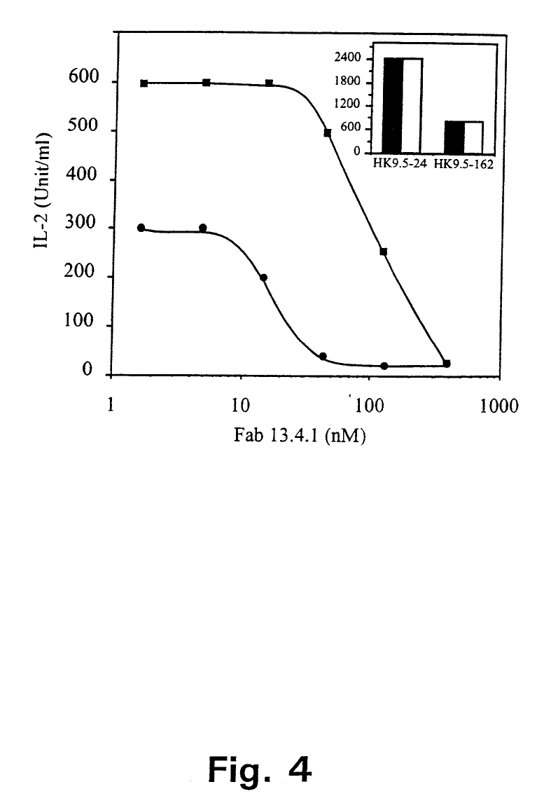

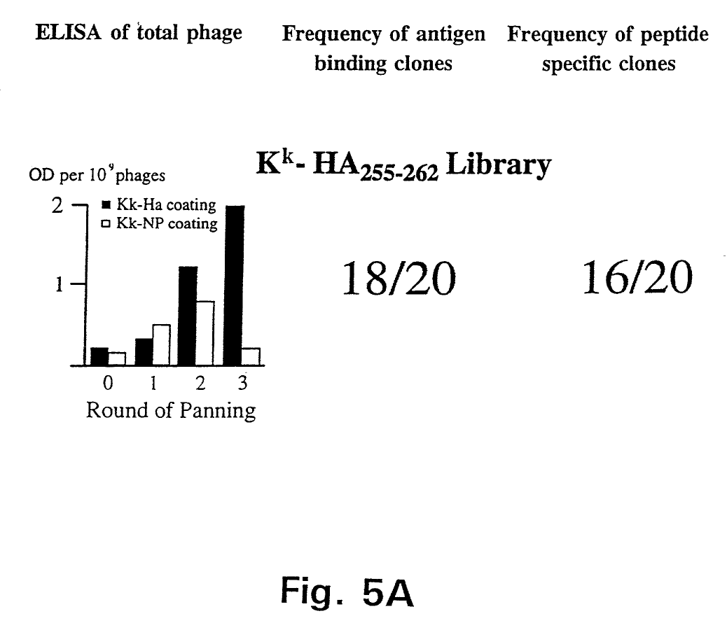

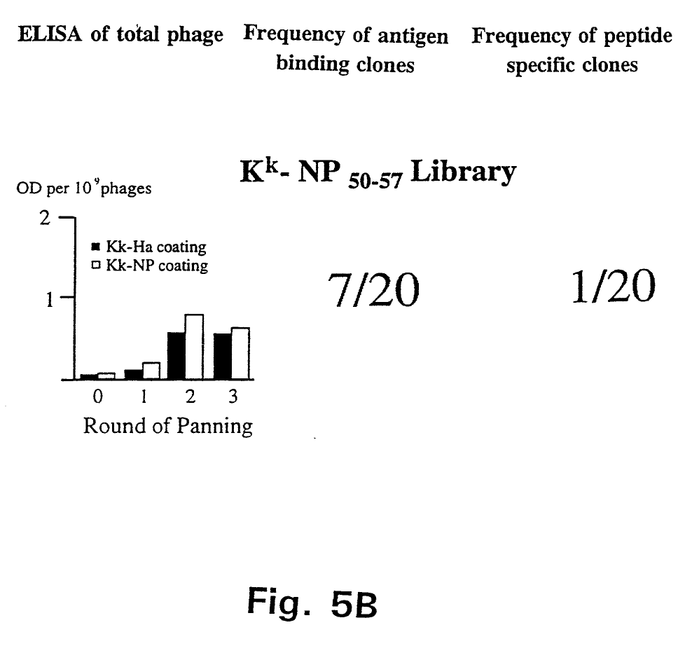

Syngenic animals were chosen to favour the generation of an antibody response directed against self-MHC restricted epitopes and the BCG-PPD immunization regime originally described by Lachmann et al., 1978, was chosen for its efficiency. Total spleen mRNA was isolated 10 days after the immunization and reverse transcribed to cDNA. Specific sets of degenerate primers were used to PCR amplify the cDNA segments corresponding to the immunoglobulin Fab fragments and the fragments were subsequently cloned into the pFab5c vector (Ørum et al., 1993) and expressed in fusion with the minor viral coat protein pIII of the filamentous bacteriophage. The initial library consisted of 2×10⁷ phages which were subjected to panning procedures followed by elution of bound phages and reamplification in E. coli. To enhance the efficiency of the selection procedure phages were panned on K^{k} transfected RMA-S cells (RMA-S·K^{k}) pulsed with Ha₂₅₅₋₂₆₂, alternating with panning on Latex beads coated with purified homogeneous Ha₂₅₅₋₂₆₂-K^{k} complexes. The purpose of the panning strategy was to change the matrix for every other panning round while maintaining a common selecting epitope, in casu Ha₂₅₅₋₂₆₂-K^{k}. Reamplified phages from each round of selection were adjusted to 2×10¹⁰ cfu/ml and tested for binding to Ha₂₅₅₋₂₆₂ or NP₅₀₋₅₇ pulsed RMA-S·K^{k} cells by FACS analysis using FITC conjugated antiphage antibodies as detecting antibody (the peptide NP₅₀₋₅₇ peptide used for comparison is also K^{k}-restricted). As illustrated in FIG. 1, a progressive enrichment for Ha₂₅₅₋₂₆₂-K^{k} recognizing Fab-phages was observed after the 2×2 alternating rounds of panning. The relative small increase in binding to NP₅₀₋₅₇ pulsed RMA-S·K^{k} cells (black bars) during panning rounds one to three probably reflects an increase in Fab phages binding with low affinity binders to common cell epitopes. This population is effectively removed as a result of the fourth and final panning round on beads.

### Example 1a

Using a similar technology to that described with respect to the antibody-Fab fragment (pSAN 13.4.1) described in detail above, other antibodies (e.g. pSAN KH04) with the same specificity of pSAN 13.4.1 but with lower affinities have been isolated. Furthermore, antibody Fab-fragments specifically recognizing the MHC-K^{k} molecule complexed with the NP₅₀₋₅₇ peptide (e.g. pSAN KN36) have been isolated. This latter antibody was isolated from an antibody Fab-library generated by the described procedure using mice immunized with the K^{k}-NP₅₀₋₅₇ complex as starting material. With respect to the latter two antibodies the approach used during the alternating panning rounds was slightly different from the one already described: Instead of alternating between pannings on plastic surfaces coated with the selected MHC/peptide complex and cells expressing the same complex, the Fab-phage library (generated from K^{k}/Ha₂₅₅₋₂₆₂ immunized mice) was panned on plastic surface coated with K^{k}/Ha₂₅₅₋₂₆₂ complexes in the presence of competing amounts of Latex beads coated with K^{k}-NP₅₀₋₅₇ complexes. Likewise, the Fab-phage library generated from K^{k}-NP₅₀₋₅₇ immunized mice was panned on plastic coated K^{k}-NP₅₀₋₅₇ complexes in the presence of Latex beads coated with K^{k}/Ha₂₅₅₋₂₅₇ complexes. It is anticipated that several versions of alternating panning strategies can be used successfully. Importantly, the applicability and generality of our immunization and alternating panning procedures have been demonstrated.

### Example 2

### Specificity of Clone pSAN 13.4.1

Individual phages were isolated from the population of phages from the final panning round and rescreened for specificity using an ELISA based assay. Of 50 clones tested, seven reacted specifically with Ha₂₅₅₋₂₆₂-K^{k} complexes (data not shown).

Fifteen clones bound to both Ha₂₅₅₋₂₆₂-K^{k} and NP₅₀₋₅₇-K^{k} complexes and most likely recognized epitopes shared by the two complexes. The remaining 28 clones bound neither to Ha₂₅₅₋₂₆₂-K^{k} or to NP₅₀₋₅₇-K^{k} complexes. The DNA sequence corresponding to the CDR3 heavy and light chain regions of these seven clones ere determined and found to be identical (data not shown) suggesting that they were all derived from one single productive antibody light chain/heavy-chain combinatorial event. Soluble Fab molecules from one of the clones, pSAN13.4.1, were produced and purified (FIG. 2A) and the specificity assayed by FACS analysis using Latex particles coated with various combinations of peptide and MHC. The specificity of the Fab molecule (Fab13.4.1) had the hallmarks of MHC-restricted T cell specificity since reactivity was only seen for complexes formed between Ha₂₅₅₋₂₆₂ and K^{k}; neither component could be recognized alone nor could NP₅₀₋₅₇-K^{k} complexes be recognized (FIG. 2B). Furthermore, the interaction was saturable and specific as evidenced by the ability of soluble Ha₂₅₅₋₂₆₂-K^{k} complexes, but neither isolated Ha₂₅₅₋₂₆₂ peptide, isolated K^{k} nor NP₅₀₋₅₇-K^{k} complexes, to compete with bead-coated Ha₂₅₅₋₂₆₂-K^{k} in binding to Fab13.4.1 (FIG. 2C). Ten to 100 nM soluble Ha₂₅₅₋₂₆₂-K^{k} complexes were needed to inhibit the interaction between Fab13.4.1 and Ha₂₅₅₋₂₆₂-K^{k} indicating that the affinity, K_{D}, of Fab13.4.1 for Ha₂₅₅₋₂₆₂-K^{k} complexes is between 10 and 100 nM.

### Example 2a

### MHC class I K^{k} Recognition of the Ha₂₅₅₋₂₆₂ Epitope

To determine the specificity of K^{k} a complete set of singly amino acid substituted peptide analogs of the K^{k}-restricted, cytotoxic T cell epitope from Influenza virus Haemagglutinin, Ha₂₅₅₋₂₆₂ (FESTGNLI) was synthesized and examined. For every position within this epitope, the parental amino acid was replaced one by one with each of the other naturally occurring amino acids (except cysteine). These analogs were tested for K^{k}-binding in a biochemical inhibition assay (25) and the effect of each substitution was expressed as -log(IC₅₀(analog)) (data not shown). Strikingly, the majority of the substitutions were more or less accepted by K^{k}. In particular, K^{k} appeared indifferent to substitutions in positions 3, 4 and 6 as none of the substitutions at these positions led to significant changes in K^{k} binding. Only 14 (or 10%) of the 144 substitutions led to more than a 100 fold drop in binding. These deleterious substitutions were entirely concentrated in positions 2 and 8, which preferred the amino acids E and I, respectively. The data suggest that position 8 is the most critical of the two since 9 substitutions led to a more than 10-fold drop in binding compared to only 6 substitutions at position 2. Less pronounced effects of substitutions were found for positions 1, 5 and 7 where F appeared to be preferred in position 1, charged amino acids and Y to be disfavored in position 5, and F and L to be preferred in position 7.

### Example 2b

### T cell Recognition of the K^{k}-restricted Ha₂₅₅₋₂₆₂ Epitope

To examine the fine specificity of MHC class I restricted T cell responses. the stimulatory capacities of each of the single amino acid substituted analogs were determined in a bioassay using the two Ha₂₅₅₋₂₆₂ specific, K^{k}-restricted T cell hybridomas, HK8.3-6F8 and HK8.3-5H3. It should be noted that this assay depends on both K^{k}-binding and T cell recognition. A dose-response analysis was made for each analog and used to determine the concentration of analogue needed to stimulate a response of 100 U/ml IL-2. The most critical peptide residues were F in position 1 (both hybridomas), T in position 4 (both hybridomas) and N in position 6 (HK8.3-6F8). In these positions, the T cell specificities were very stringent as only the parental amino acids were accepted; any substitution led to a complete loss of stimulatory activity (except for the conservative substitution of T→S in position 4, which led to a 10 fold loss). Considerable, but less stringent, specificity was observed for position 3 (both hybridomas), position 6 (HK8.3-5H3), and position 7 (both hybridomas). Only a few substitutions, mostly conservative or semi-conservative, were allowed in these positions. A slightly more promiscuous specificity was observed for position 5 (both hybridomas). Finally, the least critical peptide residues were the anchor positions for K^{k}: I in position 8 (both hybridomas) followed by E in position 2 (both hybridomas). Several substitutions were more or less accepted in these positions, and many of the substitutions, which led to a loss of T cell stimulatory activity, could be accounted for by loss in K^{k} binding.

### Example 2c

### Recognition of the K^{k} Ha₂₅₅₋₂₆₂ Epitope by the pSAN13.4.1 Antibody

PSAN 13.4.1 recognizes complexes consisting of Ha₂₅₅₋₂₆₂ and K^{k} in a way that appears reminiscent of a peptide-specific, MHC-restricted T cell specificity. To further investigate this point, the pSAN 13.4.1-binding capacity of each of the single amino acid substituted analogs was determined in a biochemical assay, in which soluble analog-K^{k} complexes were used to inhibit the binding of pSAN 13.4.1 to immobilized Ha₂₅₂₋₂₆₂-K^{k} complexes. The analog-K^{k} complexes were generated under peptide-saturating conditions to minimize the influence of K^{k} specificity and focus on the specificity of pSAN 13.4.1. The most critical residue was T in position 4, which could only be replaced with the conservative substitution, S. The second most critical residues were L in position 7 and S in position 3, where some substitutions, mostly conservative to semi-conservative, were accepted (data not shown). Less critical was F in position 1, which could be replaced by about 10 substitutions mostly conservative or semi-conservative leading to less than a 10 fold loss in binding. In contrast, the less critical residues were G in position 5 and E in position 2, which could be replaced by 13-14 amino acids. Finally, the least critical residue was I in position 8. which could be replaced with any other amino acid (except R).

Using the same peptide-MHC complexes, the fine specificity of two Ha₂₅₅₋₂₆₂-specific K^{k}-restricted T cells, and of a unique antibody, pSAN, specific for the same peptide-MHC complex has thus been determined. A striking similarity between the specificity of the T cells and that of the pSAN antibody was found and most of the peptide residues, which could be recognized by the T cells, could also be recognized by the antibody.

### Example 3

### Kinetic Binding Analysis of pSAN 13.4.1

Using surface plasmon resonance (i.e. detection of changes in refractive index on a surface; BIAcore, Pharmacia, Sweden) the specificity of Fab13.4.1 was confirmed and the kinetics of the interaction were determined. Purified Fab13.4.1 was immobilized onto BIAcore sensor chips and challenged with preformed Ha₂₅₅₋₂₆₂-K^{k} or NP₅₀₋₅₇-K^{k} complexes. Purified Fab13.4.1 was diluted to 20 μg/ml in 10 mM acetate-buffer, pH 4.5 and coupled to the dextran surface of BIAcore chips by standard amine coupling chemistry according to the manufacturer (Pharmacia, Sweden). Preformed complexes of K^{k} and peptide were made as previously described. In order to determine the concentration of formed complexes (i.e. active binding sites of K^{k}), trace amounts of iodinated NP₅₀₋₅₇ with known specific activity was added during the formation of complexes. After the separation of unbound peptide on G-25 spun columns (Stryhn et al., 1994), the amount of bound vs. free ¹²⁵I was determined by gamma counting, and the concentration of formed complexes calculated. The amount of Fab fragment coupled to the chip was kept just below 1000 RU and the flow speed was set to 10 μl/minutes, thereby avoiding interference of mass transport limitations with the kinetic calculations. All measurements were done in 0.1% Nonidet P-40 and phosphate buffered saline at 22° C.

As shown in FIG. 3A, the sensorgrams demonstrate binding of Fab13.4.1 to Ha₂₅₅₋₂₆₂-K^{k}, but not to NP₅₀₋₅₇-K^{k}. As a control, the K^{k} specific monoclonal antibody, H100-27R55 (supplied by Dr. G. Hammerling), was immobilized onto BIAcore sensor chips and challenged with same preparations of peptide-K^{k}. FIG. 3B illustrates that H100-27R55 bound both peptide-K^{k} complexes equally well confirming that equal amounts of functional K^{k} protein were available to the chips. The slope of the initial binding phase depended on the concentration of Ha₂₅₅₋₂₆₂-K^{k} (FIG. 3C) allowing us to calculate the association rate constant, kₐ, to be 1×10⁴ M⁻¹s⁻¹.

The sensorgrams of FIG. 3C revealed two experimental problems: (i) a blank injection showed an increase in signal of about 19 RU and a nearly linear decrease to about 3 RU at the end of the association time, (ii) the base line declined about 1 RU/minute during the course of the experiments, indicating a slow loss of immobilized ligand. Both these experimental deviations were interpolated into straight lines and used to correct the association phases and the entire sensorgrams, respectively. These corrections and the following calculation were done using mathematical models developed by Ron Shymko, Hagedorn Research Laboratories, Gentofte, Denmark. Twenty-one data point from each sensorgram were used for the mathematical analysis of both the dissociation and the association. First the dissociation phases were fitted by a double exponential model. The calculated k_{d} values were in the range of 7.3×10⁻⁴ s⁻¹ to 3.2×10⁻⁴ s⁻¹. Using these values and keeping the total number of binding sites constant, the entire sensorgrams were fitted using a two-site model. This gave kₐ values for the high association component in the range of 1.2×10⁴ M⁻¹s⁻¹ to 0.6×10⁴ M⁻¹s⁻¹ and the resulting K_{D} values were very similar: 53, 56, 60 and 51 nM. Parameters for the low affinity component could not be interpreted. However, the contribution of this component to the sensorgram data was small, varying from 3-18% of the total signal at the end of the association.

Removal of unbound ligand initiated a dissociation phase which could be used to determine the dissociation rate constant, k_{d}, as 5.6×10⁻⁴ s⁻¹ (corresponding to a T_{½} of about 21 minutes). The equilibrium dissociation constant, K_{D}, calculated as k_{d} divided with kₐ was found to be about 56 nM, a value, which correlates well with the inhibition range observed in FIG. 2C. Using BIAcore analysis, the kinetics and affinity of two different T cell receptors were recently published (Corr et al., 1994; Matsui et al., 1995). Association rate constants, kₐ, of 10³ and 10⁵ M⁻¹s⁻¹ and dissociation rate constants, k_{d}, of 0.06 and 0.2 s⁻¹ (corresponding to a T_{½} of 12 to 27 s) were reported leading to the calculation of overall equilibrium dissociation constants, K_{D}, of 10⁻⁵ to 10⁻⁷ M. By comparison, antibodies generally associate with about the same rate as T cell receptors, whereas they tend to dissociate orders of magnitude slower (Pellequer et al., 1993). Typically the overall affinity of antibodies are from 10⁻⁷ to 10⁻¹⁰ M. The affinity and kinetics of the Fab13.4.1 are, despite its T cell like specificity, typical of an antibody.

### Example 4

### Specific Inhibition of Antigen-specific MHC-restricted T-cell Responses

Given the specificity and affinity of Fab13.4.1 it was expected to inhibit Ha₂₅₅₋₂₆₂-specific, K^{k}-restricted T cell hybridomas, but not K^{k} restricted T cell hybridomas of different peptide specificity. Indeed, Fab13.4.1 could inhibit two Ha₂₅₅₋₂₆₂-specific, K^{k}-restricted T cell hybridomas, HK8.3-5H3 and HK8.3-6F8 (FIG. 4), whereas no effect was observed on two NP₅₀₋₅₇-specific, K^{k}-restricted, T cell hybridomas, HK9.5-24 and HK9.5-162 (Insert, FIG. 4). Hence, Fab13.4.1 recognizes a Ha₂₅₅₋₂₆₂-K^{k} dependent epitope which is spatially or allosterically related to the epitope recognized by the T cell hybridomas HK8.3-5H3 and HK8.3-6F8. The concentration of Fab13.4.1 needed to obtain inhibition of the two Ha₂₅₅₋₂₆₂-specific, K^{k}-restricted T cell hybridomas was as expected close to the K_{D} measured by the BIAcore analysis.

### Example 5

### Use of Antigen-specific MHC-restricted Antibodies for Targeting

Intriguingly, these antibodies could mimick the function of killer T cells by directing toxins to cells harbouring intracellular targets such as virus or mutated oncogenes (tumour cells) thereby eradicating those cells. These antibodies can be raised in experimental animals and subsequently “humanized”, transferred to any isotype construct and be produced recombinant in large amounts; and they might be easier to administrate and control than T cells currently in use for adoptive immunotherapy.

### References

Aharoni, R., Teitelbaum, D., Arnon, R. and Puri, J., 1991, Nature 351, 147-150

Barsomian et al., 1995, WO 95/15982

Chirgwin, J. M., Przybyla, A. E., MacDonald, R. J. and Rutter, W. J., 1979, Biochemistry 18, 5294-5299

Collier, R. J. and Caplan, D. A., 1984, Scientific American 251, 44

Corr, M., Slanetz, A. E., Boyd, L. F., Jelonek, M. T., Khilko, S., al-Ramadi, B. K., Kim, Y. S., Maher, S. E., Bothwell, A. L. and Margulies, D. H., 1994, Science 265, 946-947

Cull, M. G., Miller, J. F. and Schatz, P. J., 1992, Proc. Natl. Acad. Sci. USA 89, 1865-1869

Davies, D. R., Padlan, E. A. and Sheriff, S., 1990, Ann. Rev. Biochem. 59, 439-473

Davis, M. M. and Bjorkman, P. J., 1988, Nature 334, 395-402

Duc, H. T., Rucay P., Righenzi, S., Halle, P. O. and Kourilsky, P., 1993, Int. Immun. 5, 427-431

Dohlstein, M. et al., 1989, Proc. Natl. Acad. Sci., 91, 8945-8949

Engberg, J., Andersen, P. S., Nielsen, L. K., Dziegiel, M., Albrechtsen, and Johansen, L. K., 1995, Phage-display libraries of murine and human antibody Fab fragments, in Practical Antibody Engineering and Catalytic Antibodies (ed. Sudhir Paul), Humana Press Inc., NJ, USA, p 355-376

Fanger, M. W., Shen, L., Graziano, R. F., and Guyre, P. M., 1989, Immunol. Today 10(3), 92-99

Francisco, J. A., Campbell, R., Iverson, B. L. and Georgiou, G., 1993, Proc. Natl. Acad. Sci. USA 90, 10444-10448

Fremont, D. H., Matsumura, M., Stura, E. A., Peterson, P. A. and Wilson, I. A., 1992, Science 257, 919-927

Froscher, B. G. and Klinman, N. R., 1986, J. Exp. Med. 164, 196-210

Fugger, L., Tisch, R., Liblau, R. van Endert, P. and McDevitt H. P., 1995, The Role of Human Major Histocompatibility Complex (HLA) Genes in Disease in Scriver C. R., Beaudet, A. L., Sly, W. S. and Valle D. (eds): The Metabolic and Molecular Basis of Inherited Disease, Seventh edition, McGraw-Hill, Inc.

Griffinths, A. D., Williams, S. C., Hartley, O., Tomlinson, I. M., Waterhouse, P., Crosby, W. L., Kontermann, R. E., Jones, P. T., Low, N. M., Allison, T. J., Propero, T. D., Hoogenboom, H. R., Nissim, A., Cox, J. P. L., Harrison, J. L., Zaccolo, M., Gheradi, E. and Winter, G., 1994, EMBO J, 13(14) 3245-3260

Hammerling, DE 42 24 542 A1

Kabat E., Wu, T. T., Reid-Miller, M., Perry, H. M. and Gottesman, K. S., 1987, Sequences of Proteins of Immunological Interest, National Institute of Health, Bethesda, US Government Printing Office, Bethesda, Md.

Kaufman, D. L., Clare-Salzier, M., Tian, J., Forsthuber, T., Ting, G. S. P., Robinson, P., Athinson, M. A., Sercarz, E. E., Tobin, A. J. and Lehmann, P. V., 1993, Nature 366, 69-72

Kourilsky et al., 1991 (WO 91/12332)

Logan, J. S., 1993, Current Opinion in Biotechnology 4, 591-595

Madden, D. R., Garboczi, D. N. and Wiley, D. C., 1993, Cell 75, 693-708

Madden, D. R., 1995, The three dimensional structure of peptide-MHC complexes. Annual Review of Immunology, 13, 587-622

Matsui, K., Boniface, J. J., Steffner, P., Reay, P. A., and Davis, M. M., 1995, Proc. Natl. Acad. Sci. USA 91, 12862-12866

Michaelsson, E., Malmstrom, V., Reis, S., Engstrom, A., Burkhardt, H. and Holmdahl, R., 1994, J. Exp. Med. 180, 745-749

Morgan et al., 1994, WO 94/29451

Murphy, D. B., Lo, D., Rath, S., Brinster, R. L., Flavell, R. A., Slanetz, A. and Janeway, C. A. Jr., 1989, Nature 338, 765-768

Nemazee, D. A. and Burki, K., 1989, Nature 337, 562-565

Olsen, A. C., Pedersen, L. O., Hansen, A. S., Nissen, M. H., Olsen, M., Hansen, P. R., Holm, A. and Buus, S., 1994, Eur. J. Immunol. 24, 385-392

Puppo et al, 1995, Immunology Today 16, 124

Rubin, B., Malissen, B., Jorgensen, P. N. and Zeuthen, J., 1989, Res. Immuno. 140, 67-74

Sambrook, J., Fritsch, E. F. and Maniatis, T., 1989, Molecular Cloning. A Laboratory Manual, 2nd ed., Cold Spring Harbor Laboratory Press, Cold Spring Harbor, USA

Staerz et al., 1985, Nature 314, 628

Stryhn, A., Pedersen, L.Ø., Ortiz-Navarrete, V. and Buus, S., 1994, Eur. J. Immunol. 24, 1404-1409

Tamminen, W. L., Wraith, D. and Barber, B. H., 1987, Eur. J. Immun. 17, 999-1006

Tisch, R., Yang, X-D., Singer, S. M., Liblau, R. S., Fugger, L. and McDevitt, H. O., 1993, Nature 366, 72-75

van Leeuwen, A., Goulmy, E. and Rood, J. J., 1979, J. Exp. Med. 150, 1075-1083

van Ravenswaay-Claasen, H. H., van de Griend, R. J., Mezzanzanica, D., Bolhuis, R. L., Warnaar, S. O., and Fleuren, G. J., 1993, Int. J. Cancer 55(1), 128-136

von Boehmer, H., Teh, H. S. and Kisielow, P., 1989, Immunology Today 10, 57-61

Waterhouse, P. G., Griffiths, A. D., Johnson, K. S. and Winter, G., 1993, Combinational infection and in vivo recombination: a strategy for making large phage antibody repertoires, Nucl. Acids Res. 21, 2265-2266

Winter, G. and Milstein, C., 1991, Nature 349, 293-299

Wurcherpfennig and Strominger, 1995, J. Exp. Med., 181, 1597-1601

Wylie, D. E., Sherman, L. A. and Klinman, N. R., 1982, J. Exp. Med. 155, 403-414

Young, A. C. M., Zhang, W., Sacchettini, J. C. and Nathenson, S. G., 1994, Cell 76, 39-50

ørum, H., Andersen, P. S., Riise, E., Øster, A., Johansen, L. K., Bjørnvad, M., Svendsen, I. and Engberg, J., 1993, Nucl. Acids Res. 21(19), 4491-4498

We claim:

1. A method of producing an antibody or an antibody fragment which recognizes specific, predetermined peptide-MHC, the method comprising:
(a) producing a library expressing nucleic acids encoding an antibody or antibody fragment,
(b) selecting a unit expressing an antibody or an antibody fragment that recognizes specific, predetermined peptide-MHC complexes, and
(c) introducing the nucleic acids encoding said antibody or antibody fragment of said unit into an appropriate cell so as to produce an antibody or antibody fragment that recognizes specific, predetermined peptide-MHC complexes.

2. A method of producing an antibody or an antibody fragment that recognizes specific, predetermined peptide MHC, the method comprising:
(a) isolating RNA from an animal which has been immunized with a peptide-MHC complex,
(b) amplifying nucleic acids encoding an antibody or antibody fragment and producing a phage library expressing said nucleic acids,
(c) selecting a unit expressing an antibody or an antibody fragment that recognizes specific, predetermined peptide-MHC complexes, and
(d) introducing the nucleic acids encoding said antibody or antibody fragment of said unit into an appropriate cell so as to produce an antibody or an antibody fragment that recognizes specific, predetermined peptide-MHC complexes.

3. A method according to claim 1 wherein a selection of a unit expressing said antibody or antibody fragments with the antigen-specific MHC-restricted specificity of T-cells is performed by incubating cells expressing the peptide-MHC complex with the phage library.

4. A method according to claim 1 wherein a selection of a unit expressing said antibody or antibody fragments with the antigen-specific MHC-restricted specificity of T-cells is performed by incubating the phage library with beads to which the peptide-MHC complex is bound.

5. A method according to claim 1, wherein the selection of a unit expressing said antibody or antibody fragment that recognizes specific, predetermined peptide-MHC complexes is performed by incubating a phage library with beads to which the peptide-MHC complex is bound.

6. A method according to claim 1, wherein the selection of a unit expressing said antibody or antibody fragment that recognizes specific, predetermined peptide-MHC complexes is performed by panning a library of phages expressing antibody or antibody fragments on alternating matrixes bearing the peptide-MHC complex as the common denominator and the alternating matrixes are cells expressing the peptide-MHC complex and beads to which the peptide-MHC complex is bound.

7. A method according to claim 1 wherein purified MHC molecules that have been enriched for one particular peptide is used.

8. An antibody or antibody fragment with the antigen-specific MHC-restricted specificity of T-cells prepared by the method of claim 1.

9. A non-glycosylated antibody or antibody fragment with the antigen-specific MHC-restricted specificity of T-cells.

10. An antibody or antibody fragment according to claim 9 conjugated to a pharmaceutical wherein the pharmaceutical is selected from the group consisting of antibiotic, cytotoxic and antineoplastic agents.

11. An antibody or antibody fragment according to claim 9, wherein the antibody or antibody fragment is conjugated to a superantigen, which is capable of activating T lymphocytes, or conjugated to a polymerized carbohydrate, which is capable of activating complement.

12. A diagnostic composition for the detection of the presence of a peptide-MHC complex which comprises an antibody or antibody fragment according to claim 9.

13. A method of in vitro determining the presence of a peptide-MHC complex in an individual, comprising contacting a cell or tissue sample from said individual with a diagnostic composition which comprises an antibody or antibody fragment according to claim 9 and determining whether the fragment of antibody or antibody binds to a peptide-MHC complex in the cell or tissue sample.

14. A pharmaceutical composition for blocking an inappropriate T cell response comprising a fragment of antibody or antibody according to claim 9 and a pharmaceutical acceptable excipient.

15. A pharmaceutical composition for combating intracellularly located pathogens selected from the group consisting of viruses, bacteria and parasites, said composition comprising an antibody or antibody fragment according to claim 9 and a pharmaceutically acceptable excipient.

16. Use of a pharmaceutical composition comprising a fragment of antibody or antibody according to claim 9 and a pharmaceutically acceptable excipient for the prevention or treatment of a disease selected from the group consisting of HLA class I associated diseases (ankylosing spondylitis, Reiter disease, psoriatic spondylitis, idiopathic hemochromatosis, psoriasis vulgaris and Behcet disease) and HLA class II associated diseases (rheumatoid arthritis, pauciarticular juvenile rheumatoid arthritis, systemic lupus erythematosus, Sjogren disease, IDDM, Addison disease, Graves disease, Hashimoto disease, celiac disease, primary biliary cirrhosis, pemphigus vulgaris, epidermolysis bullosa acquisita, Hodgkin disease, cervical squamous cell circinoma, multiple sclerosis, optic neuritis, narcolepsi, myasthenia gravis, Goodpasture syndrome and alopecia areata).

17. Use of a pharmaceutical composition comprising a fragment of antibody or antibody according to claim 9 for combating intracellularly located pathogens selected from the group consisting of viruses, bacteria and parasites by directing complement lysis.

18. The method of claim 2, wherein said animal and said appropriate cell are syngenic.
